(19) United States
(12) Patent Application Publication
(10) Pub. No.: US 2003/0120267 A1
(43) Pub. Date: Jun. 26, 2003

(54) Percutaneous device for site specific delivery and method of use

(76) Inventors: Cary S. Kaufman, Bellingham, WA (US); Leslie Jacobson, Bellingham, WA (US)

Correspondence Address: BROWN, MARTIN, HALLER & MCCLAIN LLP, 1660 UNION STREET, SAN DIEGO, CA, 92101-2926, US

(21) Appl. No.: 10/136,820

(22) Filed: Apr. 30, 2002

Related U.S. Application Data
(60) Provisional application No. 60/287,001, filed on Apr. 30, 2001.
(60) Provisional application No. 60/287,006, filed on Apr. 30, 2001.
(60) Provisional application No. 60/287,007, filed on Apr. 30, 2001.
(60) Provisional application No. 60/287,009, filed on Apr. 30, 2001.
(60) Provisional application No. 60/287,012, filed on Apr. 30, 2001.

(51) Int. Cl. A61B 18/18

(52) U.S. Cl. 606/14; 606/21; 606/33

(57) ABSTRACT

The invention is a percutaneous delivery device for the delivery of a diagnostic or therapeutic agent or a physical insult to a specific location in the body. The device comprises an inner member with a preformed delivery portion that can at least partially encircle a target tissue region of interest and an outer member through which the inner member is inserted for targeting to the site of interest. The percutaneous device allows for the delivery of agents or insults from the periphery towards the center of the tumor without passage directly through the tumor or contact with the tumor.

### CROSS-REFERENCES TO RELATED APPLICATIONS

This application claims the benefit of priority of U.S. provisional applications Serial Nos. 60/287,001; 60/287,006; 60/287,007; 60/287,009 and 60/287,012 all filed Apr. 30, 2001 which are incorporated herein by reference in their entirety.

### BACKGROUND OF THE INVENTION

Surgical resection of tumors is a common therapy in the treatment of cancer. In a number of cancers, less radical surgeries have now replaced more radical measures (e.g. lumpectomy with or without lymph node resection vs. mastectomy). These newer procedures are possible due to a number of factors including improved imaging techniques that allow for the localization of the tumor such that it can be removed or destroyed without direct observation of the tumor by the surgeon. This also allows for the destruction of non-resectable tumors. Additionally less radical surgical techniques are less traumatic for the patient, resulting in a decrease in the length of hospital stay, less overall morbidity, less scarring and overall cost.

Strategies to destroy the tumor in situ have also been developed. Ablative techniques include treatment with physical insults such as radiofrequency, heat, cryotherapy or laser photocoagulation; with direct chemical insults such as ethanol, acetic acid or hot saline; or pharmacological agents such as chemotherapy, immunotherapy, and gene therapy. The insult is delivered by means of a needle inserted into the center of the tumor guided by ultrasound, computed tomography (CT), mammography, magnetic resonance imaging (MRI) or other imaging methods. For small tumors, the insult may be delivered at a single point. For larger tumors, the needle may be moved within the tumor or multiple needles may be inserted. Alternatively, probes for the delivery of physical insults may be designed with multiple tips delivered by insertion of a single needle to allow for treatment of a larger area (e.g. U.S. Pat. No. 5,928,229). A primary antenna contains at least one retractable secondary antenna to allow for repositioning of the antenna within the tissue. The secondary antenna may be an additional point source for the physical insult emitted from the antenna allowing for treatment of a larger area. Alternatively, it may be a detector to determine temperature or impedance within the tissue to monitor the ablation process.

All of the ablative techniques listed above typically involve delivery of the insult to the center of the tumor radiating outward to the periphery of the tumor. This is disadvantageous for a number of reasons. First, the cells in the center of the tumor are typically less viable and slower growing than the cells on the surface of the tumor. If the center of the tumor is already necrotic, it may protect the more superficial areas of the tumor from the insult. Second, a protective layer may be generated during the course of ablation, limiting the distribution of ablative energy and the effectiveness of tumor destruction. For example, in the case of radiofrequency (RF) ablation the tissue adjacent to the electrode becomes charred increasing impedance and limiting signal transmission through the tumor limiting its efficacy in large tumors. Third, when using laser energy, as the center of the tumor is heated, liquefaction may occur in an asymmetrical fashion, distributing ablation energy in an unpredictable fashion. Fourth, placing probes into the tumor from the center outwards can transport viable cells outwards from the tumor towards the periphery beyond the tumor promoting metastasis or peripheral implantation. After insertion of the needle, probe or antenna into the tumor for the delivery of the insult, it must be pulled through the healthy tissue for removal from the body. Any viable tumor cells adherent to the surface of the device are dragged through healthy tissue potentially seeding new tumors along the insertion path.

Eggers et al (U.S. Pat. No. 6,287,304) teach an apparatus for interstitial cauterization of tissue with electrosurgically deployed electrodes which surround the tumor. Deployment is carried out mechanically with primary electrode components using monopolar electrosurgical cutting procedures. Once so deployed the electrode assemblies carry out a biactive cauterization procedure to evoke cell death. The source electrodes are most effective on the two poles of the tumor, but have limited effectiveness on the remaining four sides. (Assuming a spherical shaped lesion has six sides in a three dimensional sense.) Although this treats from the periphery toward the center, the three dimensional distribution of energy is not achieved but rather the distribution of energy is directed from opposite poles. Thus, discrete surfaces of the tumor rather than the entire surface of the tumor is treated.

### SUMMARY OF THE INVENTION

The invention is a percutaneous delivery device for delivery of a therapeutic or diagnostic agent or physical insult to a specific location in the body. The location is at least partially encircled by the device rather than punctured, avoiding the above concerns. The device comprises a tubular sheath needle-type delivery system to temporarily or permanently place an inner member containing a pre-shaped fashioned manipulable shaped delivery portion. The inner member contains a delivery portion on the distal (first) end for the delivery of diagnostic or therapeutic materials or physical insults and a coupler on the proximal (second) end for attachment to the delivery device which is the source of the agent or insult. Delivery devices include, but are not limited to syringes, mechanical pumps and power sources. Materials to be delivered to the site of interest include, but are not limited to toxic agents such as chemotherapy, immunotherapy and gene therapy; direct molecular agents such as ethanol, diagnostic agents such as radio opaque dye material, radioisotope material or other materials that may be used in the medical field, or physical insults such as cryotherapy, laser therapy or other energy sources. The delivery portion of the inner member can be made from of any of a number of materials with the appropriate supereleastic or pseudoelastic characteristics and will vary depending on the agent to be delivered. The inner member is sharp at the distal end to facilitate passage of the delivery portion through tissue. The inner member can be solid or hollow, or combined with other material such as plastic depending upon the material or insult to be delivered. The inner member is inserted into the outer sheath through the opening at the proximal end to the site of interest at the distal end of the sheath. The delivery portion of the inner member can be deployed at a substantially different angle from the initial needle sheath puncture depending on the shape of the delivery portion and the bevel on the outer sheath. The delivery portion may contain a plurality of agent delivery portals which allow for the even discharge of the intended agent into the tissue along the predetermined orientation and shape of the delivery portion.

The invention is the use of the percutaneous delivery device to deliver physical insults or chemical agents to a specific location within the body that at least partially encircles the target site. The tumor is imaged with a three-dimensional imaging method including, but not limited to x-ray, CT, MRI and ultrasound, to allow for selection of a proper length for the outer sheath and shape for the delivery portion and to allow for proper insertion of the needle sheath. The inner member is inserted into the proximal end of the sheath and out the distal end of the sheath to a site at least partially surrounding the site of interest. The inner member may be inserted into the outer sheath either before or after insertion of the outer sheath into the body. The inner member is coupled to the delivery device and the agent or insult is delivered to the site of interest through the inner member. After delivery, the inner member is retracted into the sheath. The outer sheath, containing the inner member, is withdrawn or repositioned in the patient for administration of an agent or insult to multiple directions within a single target or multiple sites within the tissue.

The invention is a method for the amelioration and treatment of cancer using the device of the instant invention. The invention may be used alone or in conjunction with other therapies (e.g. chemotherapy, radiation, surgery, immunotherapy, hormone therapy, gene therapy ) for the treatment of cancer. Such decisions would be based on the location and stage of the disease as well as a number of other considerations well known to those skilled in the art. An individual treated with the method of the invention would be monitored to determine the efficacy of the treatment and the need for additional treatments or other therapies.

### BRIEF DESCRIPTION OF THE DRAWINGS

The present invention will be better understood from the following detailed description of an exemplary embodiment of the invention, taken in conjunction with the accompanying drawings in which like reference numerals refer to like parts and in which:

FIGS. 1A-B. A perspective view of an external sheath with a standard bevel (A) and a non-coring bevel (B).

FIG. 2. A side view of a wire inner member with a J-shaped delivery portion without additional agent delivery portals.

FIG. 3. A side view of a hollow, tubular inner member with an O-shaped delivery portion with multiple agent delivery portals.

FIGS. 4A-C. Series demonstrating the process of deployment of the inner member. A side view of the outer sheath of FIG. 1 with the delivery device inserted (A); with the delivery device partially emerged (B) and with the delivery device fully emerged (C).

FIGS. 5A-B. An enlarged sectional view taken on line 5A-5A of FIG. 3 with protruding portals (A). A sectional view with openings rather than protruding portals (B).

FIG. 6. A side view of the outer sheath of FIG. 1A with the delivery device of FIG. 2 fully inserted.

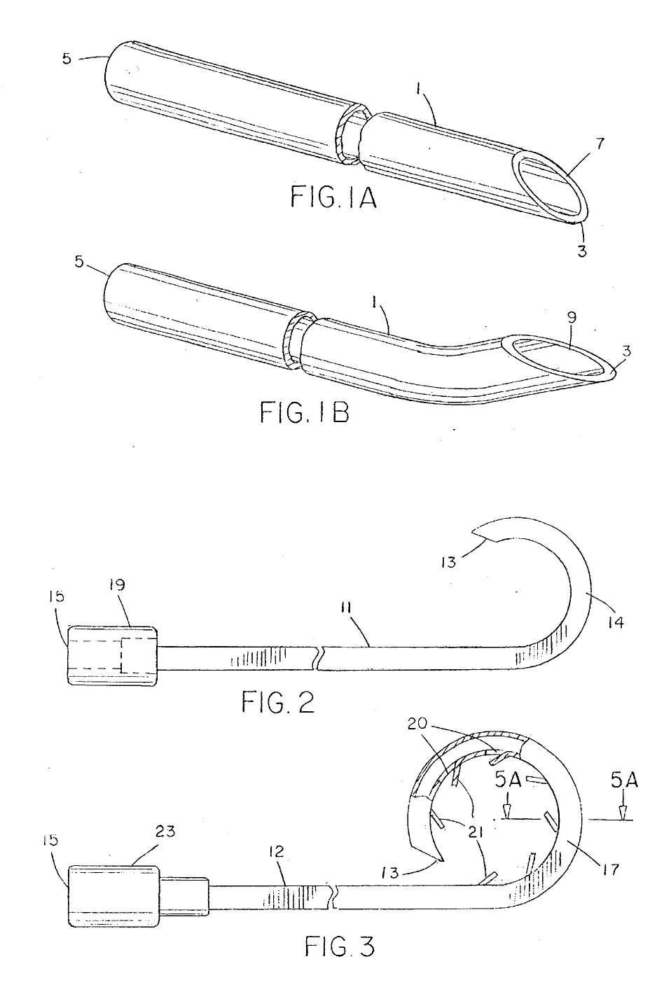

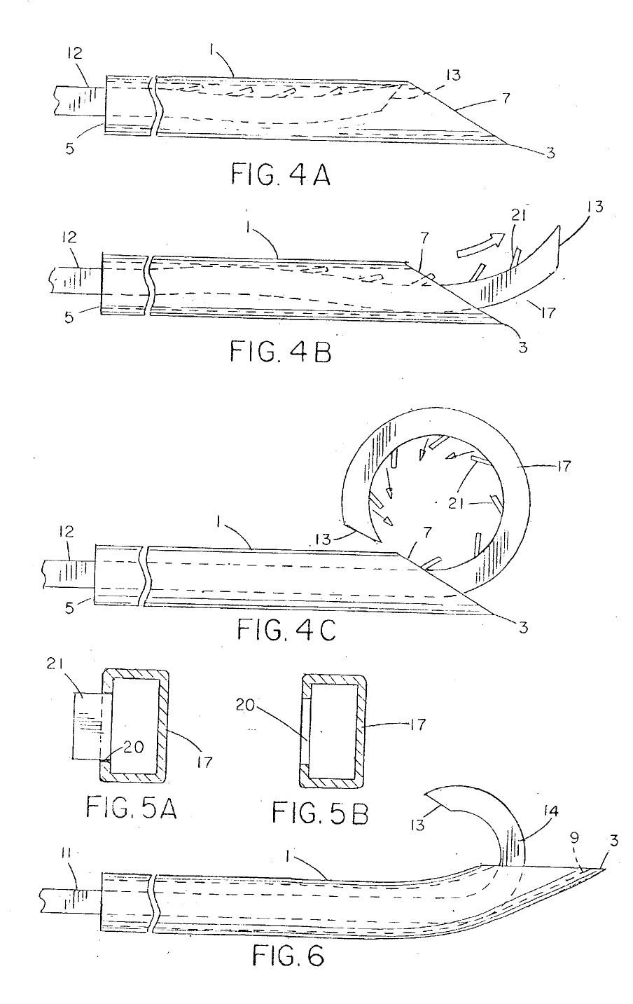

### DETAILED DESCRIPTION AND PREFERRED EMBODIMENTS

In the course of medical care, it becomes necessary to deliver certain agents to specific sites within the body. When the site is simply a small, local site, measuring no more than a few millimeters, a percutaneous needle satisfies the procedure to deliver the intended agent to the site. However, many sites of interest, such as a tumor or cancer may be a larger size. A single needle is inadequate to deliver an agent evenly throughout the site of interest. Although multiple needles may be used, this is both more difficult and inconvenient. The invention is a percutaneous device that is able to deliver the intended agent over a larger area than is possible using a single needle. Initially a needle type sheath 1 with a distal (first) end 3 which is inserted near the target site, and a proximal (second) end 5 which remains outside of the body. The distal end of the outer sheath contains either a standard 7 or non-coring bevel 9. An inner member 11 with a preformed delivery portion 17 on the distal (first) end 13 and a coupler 19 or 23 on the proximal (second) end 15 is introduced into the proximal end of the sheath 5. As the inner member 11 is introduced to the site of interest by insertion through the outer sheath 1, the delivery portion 17 of the inner member maintains a memory of the predetermined shape such that it can be deployed at a different angle than the initial needle sheath puncture. The distal end of the inner member is sharp and in a preferred embodiment, the delivery portion of the inner member is essentially flat to allow passage of the preformed portion of the inner member through tissue. Within the delivery portion of the inner member there may be a plurality of agent delivery portals that allow discharge of the agent at a plurality of sites in the region of interest along the predetermined orientation. The portals may be openings 20 or protrusions 21 in the delivery portion. Alternatively, the delivery portion may contain a number of antennae or point energy sources to deliver energy from a number of sites along the length of the delivery portion. The agent or insult is delivered by a delivery device which may include but is not limited to, a syringe, automated pump or power source which is attached to the inner member by a coupler which may include, but is not limited to, a male or female plug connector 19, a leur lock 23, a wire or wires, or fibers such as optical fibers. The inner member may be retracted into the sheath to allow repositioning or withdrawal of the needle.

The targeted tissue site is identified using three-dimensional imaging methods including, but not limited to, ultrasound, CT, MRI, stereotactic or other x-ray, Imaging also allows for the proper selection of a sheath and a delivery portion of the inner member. The delivery portion may take on any of a number of shapes including, but not limited to, C-, J-, L-, or O-shaped. Other shapes that more accurately depict the target tumor can be fashioned prior to insertion to achieve a more individualized distribution of an agent. Such shapes allow for the target tissue to be at least partially, and preferably completely, encircled. Depending on the agent or insult to be delivered, the delivery portion may contain a plurality of portals or antennae. The portals or antennae are preferably directed generally towards the center of the region being encircled.

The inner member is advanced into the needle sheath using a lead wire so the distal ends of both the inner member and outer sheath coincide. The inner member and outer sheath are designed so that when combined, their distal end appears as a single, sharp site that will pass through tissue due to its pointed shape. Such assemblies are well known. If the sheath contains a standard needle bevel (e.g. a coaxial needle), the inner member can be designed to prevent the needle from coring or an obturator can be used to place the outer tubular member at a specific location, thereafter the obturator may be exchanged for the inner member. Alternatively, the sheath may contain a non-coring needle bevel such that nothing is required to block the end upon insertion. The sheath is composed of any of a number of materials used to make needles including stainless steel, titanium, cobalt, nickle, tantalum and mixtures and alloys thereof. The outer sheath may be round or oblong depending on the shape of the inner member to be inserted therethrough. The inner member can be inserted into the sheath before or after insertion of the device into the body.

The device is placed at the edge of the target tissue with image visualization using any of a number of imaging methods. The distal end of the sheath is placed at a site outside of the tumor, but sufficiently close to allow the inner member to at least partially encircle the tumor when deployed. The desired margin around the tumor is considered when choosing the predetermined shape of the inner member along with the expected diameter of the material administration zone The material administration zone is considered the internal area within the region surrounded by the preformed delivery portion of the inner membrane. This also includes a small margin of tissue outside the perimeter of the delivery portion.

After placement of the sheath at the edge of the target tissue, the inner member is advanced under image visualization to the desired position such that it at least partially encircles the target tissue when deployed while keeping the outer member stable. Once deployed, the target tissue is treated with a physical insult or agent delivered through or by the plurality of portals or antennae in the delivery portion of the inner member surrounding the target. Thus, the surface growing edge of the tumor is treated first with the interior treated with continuous delivery of the agent. With progressive treatment the inner portions of the tumor are eventually engulfed with the agent. An alternative treatment method is to activate delivery of the agent and continue delivery as the inner member is withdrawn. After treatment, the inner member is drawn completely into the outer sheath. Both the inner and outer portions are withdrawn from the target tissue together. As no portion of the apparatus comes in contact with the tumor, cells from the tumor cannot be inadvertently transported into healthy tissue and the tumor cannot be fragmented by mechanical disruption.

The material used to make the inner member is not central to the invention. The materials may be any of a variety of stainless steels, cobalt, titanium, nickle and tantalum alloys and mixtures thereof, and other alloys typically used for their memory and hypoallergenic properties. It has been shown that certain titanium-nickel alloys are especially suitable for use with a device such as this. These materials have the ability to be transformed to exhibit superelastic and pseudoelastic characteristics (e.g. see U.S. Pat. Nos. 3,174,851 and 4,435,229). Some of these alloys are known as “nitinol”. Properties of the inner member will include this type of elasticity along with the ability to withstand the variety of levels of heating and cooling that would occur in using the method of the invention. Semi-rigid plastics may also be used to form the inner member or the preformed portion of the inner member. In addition, the inner member may be a combination of elastic material attached to a plastic catheter. The elastic material would direct the plastic inner member through the encircling path around the target area. Fluids can be delivered through the plastic inner member to the site of interest. Plastics for use in such applications may be any of a number of plastics used in intravenous apparatuses and are well known to those skilled in the art.

Size of tumor to be treated only dependent upon ability to adapt the delivery portion for various tumor shapes and sizes. This can be done most readily by altering the size and shape of the delivery portion as well as the density of portals or antennae. In larger tumors, one can have multiple delivery members within a single outer sheath, allowing the deployment of members in multiple directions.

A number of devices have been described for the delivery of various agents to the site of interest. Endoscopes for administration of microwave and RF hyperthermia are described in U.S. Pat. Nos. 4,409,993 and 4,920,978 respectively (both incorporated herein by reference). An electrode system for delivery of electrical energy is described in U.S. Pat. No. 4,565,200 (incorporated herein by reference). Such apparatuses can be modified for delivery of a variety of physical insults using the percutaneous device of the invention by methods well known to those skilled in the art. An apparatus for the delivery of a conductive fluid to a tumor is taught in U.S. Pat. No. 5,807,395 (incorporated herein by reference). Such an apparatus can be modified for the delivery of any of a number of fluids via the percutaneous needle of the invention by methods well known to those skilled in the art.

The choice of agent or insult to be delivered using the device of the invention is a matter of choice that can be readily made by one skilled in the art. Considerations regarding such a choice are dependent on the patient, the stage and location of the tumor or tumors, the previous and concurrent therapies being used to treat the patient and the efficacy of various agents in the treatment of the specific type of tumor.

### EXAMPLE 1

Delivery of chemical interventions to liver tumor tissue. The liver is a common site for metastasis for cancers from remote locations of the body. Due to the highly vascularized nature of the liver, it is difficult to resect tumors surgically. However, it is an ideal tissue for treatment using the percutaneous device of the invention. Single or multiple tumors can be identified using a CT imaging table or ultrasound. With the area of interest visualized using the imaging method of choice, the three dimensional depiction of the tumor may be identified. Based on information obtained from imaging studies, a preformed, O-shaped inner member with a plurality of portals of the appropriate size to completely encircle the tumor is selected. The inner member is inserted into the outer sheath and the device is inserted into the patient until the distal end approaches the tumor. The device may be rotated to direct the bevel before deployment of the inner member. The inner member composed of a titanium-nickel (NiTi) alloy combined with a plastic tubing preformed delivery device is deployed to encircle the tumor. Imaging confirms its position. A volume of ethanol proportional to the size of the tumor is injected through the inner member over 5 minutes exiting via multiple sites along the inner member. Injection is via hand injection or via pump attached to the inner member by a secure leur lock type of mechanism. A small amount of radio-opaque dye may be added to the ethanol or injected immediately afterwards to x-ray and image the treated area. The inner member is retracted into the outer sheath and the device is removed from the body. In the case of a large tumor, it is possible to reposition the device angled by 45 to 60 degrees before removing it from the body or to insert multiple devices to treat a single tumor. Multiple tumors can be treated by several passes of this instrument.

### EXAMPLE 2

Delivery of laser energy to breast tumor tissue at multiple sites. Tumors are localized by methods similar to those described for liver tumors. Targets are determined using three-dimensional imaging and preformed delivery devices are selected based on the size and shape of tumors to be destroyed. The outer sheath with the bevel blocked by an obturator is inserted and placed close to the tumor. The obturator is removed and the inner member with an C-shaped delivery device is inserted through the sheath and deployed around the tumor. The tumor is heated using laser energy to a level adjusted according to the size of the tumor targeted. The inner member is drawn into the outer sheath such that it is flush with the bevel at the distal end of the outer sheath to allow for repositioning of the needle without withdrawal from the patient if desired. The outer sheath can be rotated 45 to 60 degrees to reposition the device more comprehensively around the tumor. The treatment is repeated at the next site of interest until all of the sites have been treated.

Although an exemplary embodiment of the invention has been described above by way of example only, it will be understood by those skilled in the field that modifications may be made to the disclosed embodiment without departing from the scope of the invention, which is defined by the appended claims.

We claim:

1. A percutaneous delivery device comprising:
an inner member comprising a first end and a second end wherein the first end comprises a preformed delivery portion and the second end comprises a coupler for coupling the inner member to a delivery source; and
an outer sheath comprising an inner diameter, a first end and a second end, wherein the inner diameter of the sheath is sufficiently large to allow for insertion of the inner member into the second end of the outer sheath and the first end of the outer sheath is sufficiently sharp to pierce tissue and contains an opening sufficiently large for passage of the preformed delivery device.

2. The percutaneous device of claim 1, wherein the preformed delivery portion is comprised of a material that will resume a predetermined shape after insertion through the outer sheath.

3. The percutaneous device of claim 1, wherein the preformed delivery portion is comprised of a material that displays supereleastic or pseudoelastic properties.

4. The percutaneous device of claim 1, wherein the preformed delivery portion is composed of a material selected from the group consisting of titanium, nickle, cobalt, stainless steel and tantalum and mixtures and alloys thereof.

5. The percutaneous device of claim 1, wherein the preformed delivery portion is composed of a material selected from the group consisting of semi-rigid plastics.

6. The percutaneous device of claim 1, wherein the preformed delivery portion is shaped to at least partially encircle a tumor.

7. The percutaneous device of claim 1, wherein the preformed delivery portion is a shape selected from the group consisting of C-, J-, L-, or O-shaped.

8. The percutaneous device of claim 1, wherein the preformed delivery portion is a random shape.

9. The percutaneous device of claim 1, wherein the preformed delivery portion contains a plurality of portals.

10. The percutaneous device of claim 1, wherein the delivery portion of the inner member can be deployed in a direction non-parallel to the outer sheath.

11. The percutaneous device of claim 1, wherein the delivery source delivers liquid.

12. The percutaneous device of claim 11, wherein the liquid delivered is a therapeutic agent.

13. The percutaneous device of claim 11, wherein the liquid delivered is a direct molecular agent.

14. The percutaneous device of claim 11, wherein the liquid delivered is an imaging agent.

15. The percutaneous device of claim 1, wherein the delivery source delivers a physical insult.

16. The percutaneous device of claim 15, wherein the physical insult is heat.

17. The percutaneous device of claim 15, wherein the physical insult is cold.

18. The percutaneous device of claim 15, wherein the physical insult is selected from the group consisting of radiofrequency wave, laser coagulation and cryotherapy.

19. The percutaneous device of claim 1, wherein the coupler of the inner member comprises a luer lock.

20. The percutaneous device of claim 1, wherein the coupler of the inner member comprises a wire.

21. The percutaneous device of claim 1, wherein the coupler of the inner member comprises a tube.

22. The percutaneous device of claim 1, wherein the coupler of the inner member comprises a fiber.

23. The percutaneous device of claim 1, wherein the outer sheath is at least partially radio-opaque.

24. The percutaneous device of claim 1, wherein the outer sheath is composed of a material selected from the group consisting of titanium, nickle, cobalt, stainless steel and tantalum and mixtures and alloys thereof.

25. The percutaneous device of claim 1, wherein the opening on the first end of the outer sheath is a bevel.

26. The percutaneous device of claim 25, wherein the bevel is a standard bevel.

27. The percutaneous device of claim 25, wherein the bevel is a non-coring bevel.

28. A method for the treatment of cancer comprising:
localizing a tumor in an individual by a three-dimensional imaging technique,
inserting a percutaneous delivery device adjacent to the tumor, the percutaneous delivery device comprising an inner member comprising a first end and a second end wherein the first end comprises a preformed delivery portion and the second end comprises a coupler for coupling the inner member to a delivery source and
an outer sheath comprising an inner diameter, a first end and a second end, wherein the inner diameter of the sheath is sufficiently large to allow for insertion of the inner member into the second end of the outer sheath and the first end of the outer sheath is sufficiently sharp to pierce tissue and contains an opening sufficiently large for passage of the preformed delivery device;
deploying the inner member to at least partially encircle the tumor with the preformed delivery device;
delivering an insult or agent to the tumor; and
withdrawing the delivery device into the sheath for repositioning of the percutaneous device.

29. The method of claim 28, wherein additional agent or insult is delivered after repositioning of the percutaneous device.

30. The method of claim 28, where repositioning comprises removal from the percutaneous device from the individual.
